(19) United States
(12) Patent Application Publication
(10) Pub. No.: US 2024/0016720 A1
(43) Pub. Date: Jan. 18, 2024

(54) REDUCTION OF RANCIDITY OF VEGETABLE OILS

(71) Applicant: DSM IP Assets B.V., HEERLEN (NL)

(72) Inventors: Cyrille DESHAYES, Kaiseraugst (CH); Christine MENDROK-EDINGER, Kaiseraugst (CH)

(21) Appl. No.: 18/256,401

(22) PCT Filed: Dec. 8, 2021
(86) PCT No.: PCT/EP2021/084826

§ 371 Date: Jun. 7, 2023

(30) Foreign Application Priority Data
Dec. 9, 2020 (EP) 20212808.8

(51) Int. Cl. A61K 8/73; A61K 8/31; A61K 8/92

(52) U.S. Cl. CPC A61K 8/732; A61K 8/31; A61K 8/922

(57) ABSTRACT

The present invention relates to the reduction of rancidity of vegetable oils which are prone to get rancid by adding an ester of a fatty acid and dextrin and/or a mixture of branched and linear saturated C15-C19 alkanes. This allows to obtain a longer shelf-life of these vegetable oils and its cosmetic composition without 5 adding antioxidants.

### TECHNICAL FIELD

The present invention relates to the rancidity of vegetable oils.

### BACKGROUND OF THE INVENTION

Vegetable oils are prone to get rancid. Rancidity refers to the complete or incomplete hydrolysis or oxidation of fats and oils when exposed to air, light, moisture, and bacterial activity; this generally occurs in food items making them undesirable for consumption. Rancid vegetable oils have a typical odd smell and/or taste. Once the oil has turned rancid, there is no way to go back and fix it.

Also cosmetic compositions contain vegetable oils, on the one hand to provide special cosmetic effects on skin and on the other hand to make the products more natural. Because sustainability is also in the cosmetic industry an increasing trend, more and more cosmetic formulations contain vegetable oils.

As the odd smell of rancid oils is very intensive, already small amounts of rancid oils render a cosmetic composition as inacceptable by any customers.

Vegetable oils based on a high degree of unsaturated fatty acids are known to be much more prone to rancidity than the respective oils based on saturated fatty acids.

Usually antioxidants are used to reduce the rancidity of vegetable oils, i.e. to reduce the susceptibility of vegetable oils to get rancid, or to extent the time until the vegetable oil gets rancid.

Cosmetic composition are very sensitive to smell, particular to smell changes. The customers particularly don't accept any cosmetic products which have an odd smell resulting from rancid ingredients.

As vegetable oils are broadly used in cosmetic composition due to their beneficial effects to hair and skin, the rancidity is a big problem which needs to be reduced in the formulation of a cosmetic composition.

### SUMMARY OF THE INVENTION

Therefore, the problem to be solved by the present invention is to reduce the rancidity of cosmetic composition comprising vegetable oils based on a high amount of unsaturated fatty acids.

Surprisingly, it has been found that the cosmetic composition according to claim 1 solves this problems. It has been particularly found that an ester of a fatty acid and dextrin and/or a mixture of branched and linear saturated C15-C19 alkanes strongly reduce the rancidity of vegetable oils in general and of vegetable oils based on a high amount of unsaturated fatty acid in particular. Very surprisingly, it has been found that the combination of ester of a fatty acid and dextrin and a mixture of branched and linear saturated C15-C19 alkanes reduced the rancidity particularly strongly.

Reduction of rancidity results in a longer shelf-life of the vegetable oil or the cosmetic composition, respectively.

Further aspects of the invention are subject of further independent claims. Particularly preferred embodiments are subject of dependent claims.

### DETAILED DESCRIPTION OF THE INVENTION

In a first aspect the present invention relates to a cosmetic composition comprising
an ester of a fatty acid and dextrin
a mixture of branched and linear saturated C15-C19 alkanes
a vegetable oil having an amount of unsaturated fatty acids of more than 35% by weight, particularly more than 40% by weight, in its triglycerides;

wherein the amount of branched saturated C15-C19 alkane in said mixture of branched and linear saturated C15-C19 alkanes is more than 80% by weight, preferably more that 90% by weight, most preferred more than 92% by weight.

For sake of clarity, some terms used in the present document are defined as follows:

In the present document, a “Cₓ-C_{y} alkane” is an alkane comprising x to y carbon atoms, i.e., for example, a C15-C19 alkane is an alkane comprising 15 to 19 carbon atoms. The alkane can be linear or branched (i.e. non-linear) and are purely saturated hydrocarbons. For example, all alkanes having the molecular formula C₁₅H₃₂, C₁₆H₃₄, C₁₇H₃₆, C₁₈H₃₈ and C₁₉H₄₀, such as pentadecane, octadecane, nonadecane, 2,6,10,14-tetramethylpentadecane, isohexadecane, are regarded as C15-C19 alkanes. Particularly preferred branched alkanes are branched alkanes having exclusively methyl group(s) as side chain(s), such as e.g. 2,6,10,14-tetramethylpentadecane, 2-methylpentadecane or 3-methylpentadecane.

In case identical labels for symbols or groups are present in several formulae, in the present document, the definition of said group or symbol made in the context of one specific formula applies also to other formulae which comprises the same said label.

The term “UV filter” in the present document stands for a substance that absorbs ultraviolet light (=UV light), i.e. electromagnetic radiation of the wavelength between 280 and 400 nm. UV(A) filters are UV filters that absorb UV(A) light, i.e. electromagnetic radiation of the wavelength between 315 and 400 nm. UV(B) filters are UV filters that absorb UV(B) light, i.e. electromagnetic radiation of the wavelength between 280 and 315 nm.

A liquid organic UV filter is liquid at ambient temperature (i.e. 25° C.).

A solid organic UV filter is solid at ambient temperature (i.e. 25° C.).

A “mixture of branched and linear saturated C15-C19 alkanes” in the present document means that said mixture comprises different alkanes each of them only having 15, 16, 17, 18 or 19 carbon atoms but does not comprise any alkanes having less carbons. Therefore, such a mixture does not contain for example dodecane or isododecane. Said mixture comprises both branched and linear C15-C19 alkanes.

### Vegetable Oil

The cosmetic composition comprises a vegetable oil. Vegetable oil are extracted from plants, particular from seeds and less often from other parts of fruits. Vegetable oils are preferably liquid at room temperature and are usually edible.

A vegetable oil in this document mainly consists of triglycerides. The triglycerides are esters of glycerol with fatty acids. The fatty acid can be saturated or unsaturated.

The cosmetic composition comprises a vegetable oil having an amount of unsaturated fatty acids of more than 35% by weight, particularly more than 40% by weight, in its triglycerides.

The term “fatty acids in its triglyceride” relates in this document to the part of the glyceride of a vegetable oil which in its formula originates from the respective acid being esterified with the glycerol.

Vegetable oils based on a high degree of unsaturated fatty acids are known to be much more prone to rancidity than the respective oils based on saturated fatty acids.

So for example, linseed oil having more than 80% unsaturated fatty acid in its triglycerides is much more prone to get rancid than other oils having a high amount of saturated fatty acids in its triglyceride.

It is preferred that the ratio of the weight of mono-unsaturated fatty acids to the weight of unsaturated fatty acids in its triglycerides is more than 80%, by weight, preferably more than 85% by weight, most preferably more than 90% by weight.

The vegetable oil is preferably an oil being selected from the group consisting of Macadamia integrifolia seed oil, Prunus amygdalus dulcis (sweet almond) oil, Argania spinosa kernel oil, Linum usitatissimum (linseed) oil, Perilla ocymoides seed oil, Butyrospermum parkii (shea) butter and Olea europaea (Olive) fruit oil.

It is particularly preferred that the vegetable oil is selected from the group consisting of Macadamia integrifolia seed oil, Butyrospermum parkii (shea) butter and Olea europaea (Olive) fruit oil.

### Ester of a Fatty Acid and Dextrin

The cosmetic composition further comprises an ester of a fatty acid and dextrin.

Dextrin is an oligomer polymers of D-glucose. Its structure can be represented simplified by the following structure

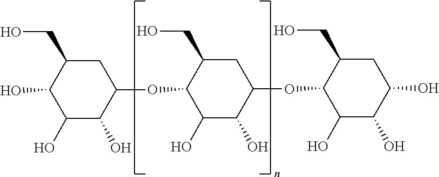

Dextrins have different average degrees of glycopolymerization which leads to different molecular weights.

In the present invention, the dextrin of said ester of a fatty acid and dextrin has preferably an average degree of glycopolymerization of between 3 and 20, particularly between 8 and 16.

It is preferred that the fatty acid of said ester of a fatty acid and dextrin is a C14-C18 fatty acid, particularly a linear C14-C18 fatty acid, most preferably palmitic acid.

As particular suitable ester of a fatty acid and dextrin is a dextrin palmitate as commercialized as Rheopearl® KL2 by Chiba Flour Milling.

Dextrin has several hydroxyl groups which can be esterified.

It is preferred that said ester of a fatty acid and dextrin has an average number of esterified hydroxyl groups of more than 2.5, preferably between 2.7 and 3.5, more preferably between 28 and 3.4, most preferably between 2.8 and 3.2, per glucose unit.

In one embodiment said ester of a fatty acid and dextrin has an average number of esterified hydroxyl groups of more than 3, preferably between 3.05 and 3.5, more preferably between 3.1 and 3.4, most preferably between 3.1 and 3.2, per glucose unit.

In other words, preferably essentially all of the hydroxyl groups of the dextrin are esterified.

It is further preferred that said ester of a fatty acid and dextrin has an molecular weight M n of between 8′000 and 16′000 Da, preferably between 9′000 and 13′000 Da, more preferably between 10′000 and 11′500 Da.

The molecular weight Mn is determined in Dalton (Da) particularly by SEC/GPC using polystyrene as standard.

Both fatty acid and dextrin have biological origin. The biological origin of chemicals is very advantageous as such material or products thereof have a high degree of sustainability. High sustainable products or compositions are highly demanded in the market.

### Mixture of Branched and Linear Saturated C15-C19 Alkanes

The cosmetic composition comprises a mixture of branched and linear saturated C15-C19 alkanes.

Particular suitable mixtures of C15-C19 alkanes are particularly the ones disclosed in WO 2016/185046, WO 2017/046177, WO 2018/109353 A1 and WO 2018/109354 A1 and WO 2018/172228 A1.

Preferably, the mixture of branched and linear saturated C15-C19 alkanes has a content of carbon of biological origin being greater or equal to 90% with respect of the total weight of the mixture of branched and linear saturated C15-C19 alkanes. The biological origin of chemicals is very advantageous as such material has a high degree of sustainability. High sustainable products or compositions are highly demanded in the market.

The determination of the content of biomaterial or content of biocarbon is given pursuant to standards ASTM D 6866-12, method B (ASTM D 6866-06) and ASTM D 7026 (ASTM D 7 026-04). Standard ASTM D 6866 concerns “Determining the Biobased Content of Natural Range Materials Using Radiocarbon and Isotope Ratio Mass Spectrometry Analysis”, while standard ASTM D 7 026 concerns “Sampling and Reporting of Results for Determination of Biobased Content of Materials via Carbon Isotope Analysis”. The second standard mentions the first in its first paragraph. The first standard describes a test of measurement of the ratio ¹⁴C/¹²C of a sample and compares it with the ratio ¹⁴C/¹²C of a sample renewable reference of origin 100%, to give a relative percentage of C of origin renewable in the sample. The standard is based on the same concepts that the dating with ¹⁴C.

It is further preferred that the composition has no or a very small amount (less than 100 ppm, particularly less than 30 ppm) of aromatic hydrocarbons with respect to the total weight of the mixture of branched and linear saturated C15-C19 alkanes.

The mixture of branched and linear saturated C15-C19 alkanes is particularly produced by catalytic hydrogenation of hydrocarbon biomass feedstock, such as described in detail in WO 2016/185046, particular the one disclosed as example 3 of WO 2016/185046.

It is preferred that the amount of linear saturated C15-C19 alkanes in said mixture of branched and linear saturated C15-C19 alkanes is less than 10% by weight, preferably less than 8% by weight, most preferred more than 5% by weight.

It is further preferred that the amount of C15 is less than 3%, particularly less than 1%, preferably less than 0.05%, by weight in respect to the weight of said mixture of branched and linear saturated C15-C19 alkanes.

It is preferred that the mixture of branched and linear saturated C15-C19 alkanes is a mixture of branched and linear saturated C16-C19 alkanes.

It is further preferred that the amount of branched saturated C16-C18 alkane is more than 90% by weight, preferably more than 95% by weight in respect to the weight of said mixture of branched and linear saturated C15-C19 alkanes.

It is further preferred that the amount of C15 alkanes is less than 5%, particularly less than 2%, by weight in respect to the weight of said mixture of branched and linear saturated C15-C19 alkanes.

It is further preferred that the amount of branched saturated C17-C18 alkane is more than 85% by weight, preferably more than 92% by weight in respect to the weight of said mixture of branched and linear saturated C15-C19 alkanes.

It is further preferred that the amount of C17 alkanes is more between 15 and 20% by weight in respect to the weight of said mixture of branched and linear saturated C15-C19 alkanes.

It is further preferred that amount of branched saturated C18 alkane is more than 50% by weight, preferably more than 60% by weight, even more preferably more than 70% by weight, relative to the weight of said mixture of branched and linear saturated C15-C19 alkanes.

It is further preferred that the amount of C18 alkanes is particularly between 70 and 75% by weight in respect to the weight of said mixture of branched and linear saturated C15-C19 alkanes.

In other words, the mixture of branched and linear saturated C15-C19 alkanes consist preferably mainly of C18 alkane(s), most preferably mainly of branched C18 alkane(s).

As the cosmetic composition comprises a mixture of branched and linear saturated C15-C19 alkanes, said composition does not comprise any lower alkanes, i.e. it does particularly not comprise any C12 alkanes and particularly does not comprise any C12 or C13 or C14 alkanes.

It is further preferred that the mixture of C15-C19 alkanes has at 20° C., a viscosity of 3-15 mPa·s, particularly between 6 and 12 mPa·s.

It is further preferred that the mixture of C15-C19 alkanes has at 20° C. a refractive index of between 1.40 and 1.48, particularly of between 1.42 and 1.45, most preferably between 1.43 and 1.44.

It is further preferred that the mixture of C15-C19 alkanes is the mixtures of C15-C19 alkanes as commercialized as EMOGREEN™ L19 by SEPPIC

In the said composition the ratio of the weight of said ester of a fatty acid and dextrin to the weight of said mixture of branched and linear saturated C15-C19 alkanes is preferably less than 100% by weight, preferably in the range of 50-80% by weight, most preferred in the range of 60 -70% by weight.

In other words, the composition comprises preferably more, by weight, of the C15-C19 alkanes than of the ester of a fatty acid and dextrin.

It is preferred that the mixture of branched and linear saturated C15-C19 alkanes and the a fatty acid and dextrin are both based on organic origin.

It is furthermore preferred that the mixture of branched and linear saturated C15-C19 alkanes and the ester of a fatty acid and dextrin are readily biodegradable according to OECD 301B.

The cosmetic composition typically comprises other ingredients which are suitable for the use in cosmetic compositions.

The cosmetic composition comprises preferably water.

The cosmetic compositions may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (0/W/0-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

Preferred cosmetic compositions in all embodiments of the present invention comprise water and are in the form of an emulsion.

The emulsion particularly contain an oily phase and an aqueous phase such as in particular O/W, W/O, Si/W, W/Si, O/W/O, W/O/W multiple or a pickering emulsions.

The total amount of the oily phase present in such emulsions is preferably at least 10 wt.-%, such as in the range from 10 to 60 wt.-%, preferably in the range from 15 to 50 wt.-%, most preferably in the range from 15 to 40 wt.-%, based on the total weight of the cosmetic composition.

The amount of the aqueous phase present in such emulsions is preferably at least 20 wt. %, such as in the range from 40 to 90 wt.-%, preferably in the range from 50 to 85 wt.-%, most preferably in the range from 60 to 85 wt.-%, based on the total weight of the cosmetic composition.

More preferably, the cosmetic compositions are in the form of an oil-in-water (O/W) emulsion comprising an oily phase dispersed in an aqueous phase in the presence of an O/W- respectively Si/W-emulsifier. The preparation of such emulsions is well known to a person skilled in the art.

The compositions in form of O/W emulsions can be provided, for example, in all the formulation forms for O/W emulsions, for example in the form of serum, milk or cream, and they are prepared according to the usual methods. The compositions are preferably intended for topical application and can in particular constitute a dermatological or cosmetic composition, for example intended for protecting human skin against the adverse effects of UV radiation (antiwrinkle, anti-ageing, moisturizing, sun protection and the like).

The cosmetic composition may further comprise also organic or inorganic UV filters known to the person skilled in the art of cosmetics and sun protection.

Particularly, the cosmetic composition further comprises at least one liquid organic UV filter and/or at least one solid organic UV filter.

Suitable liquid organic UV-filter absorb light in the UV(B) and/ or UV(A) range and are liquid at ambient temperature (i.e. 25° C.). Such liquid UV-filter are well known to a person in the art and encompass in particular cinnamates such as e.g. octyl methoxycinnamate (PARSOL® MCX) and isoamyl methoxycinnamate (Neo Heliopan® E 1000), salicylates such as e.g. homosalate (3,3,5 trimethyl-cyclohexyl 2-hydroxybenzoate, PARSOL® HMS) and ethylhexyl salicylate (also known as ethylhexyl salicylate, 2-ethylhexyl-2-hydroxybenzoate, PARSOL® EHS), acrylates such as e.g. octocrylene (2-ethylhexyl-2-cyano-3,3-diphenylacrylate, PARSOL® 340) and ethyl 2-cyano-3,3 diphenylacrylate, esters of benzalmalonic acid such as in particular dialkyl benzalmalonates such as e.g. di (2-ethylhexyl) 4-methoxybenzalmalonate and polysilicone 15 (PARSOL® SLX), dialkylester of naphthalates such as e.g. diethylhexyl 2,6-naphthalate (Corapan® TQ), syringylidene malonates such as e.g. diethylhexyl syringylidene malonate (Oxynex® ST liquid) as well as benzotriazolyl dodecyl p-cresol (Tinoguard® TL) as well as benzophenone-3 and drometrizole trisiloxane.

Particular advantageous liquid organic UV-filter are octyl methoxycinnamate, homosalate, ethylhexyl salicylate, octocrylene, diethylhexyl 2,6-naphthalate, diethylhexyl syringylidene malonate, benzotriazolyl dodecyl p-cresol, benzophenone-3, drometrizole trisiloxane as well as mixtures thereof.

In a preferred embodiment, the liquid UV filter is a liquid UV(B) filter which is selected from the group consisting of ethylhexyl methoxycinnamate, octocrylene, homosalate, ethylhexyl salicylate, benzophenone-3 and drometrizole trisiloxane.

Suitable solid organic UV-filter absorb light in the UV(B) and/ or UV(A) range and are solid at ambient temperature (i.e. 25° C.). Particularly suited solid UV-filters are of the group consisting of bis-ethylhexyloxyphenol methoxyphenyl triazine, butyl methoxydibenzoyl methane, methylene bis-benzotriazolyl tetramethylbutylphenol, diethylamino hydroxybenzoyl hexyl benzoate, ethylhexyl triazone, diethylhexyl butamido triazone, 4-methylbenzylidene camphor and 1,4-di(benzoxazol-2′-yl)benzene.

A preferred solid organic UV(A) filter is a UV(A) filter which is selected from the group consisting of bis-ethylhexyloxyphenol methoxyphenyl triazine, butyl methoxydibenzoyl methane, methylene bis-benzotriazolyl tetramethylbutylphenol and diethylamino hydroxybenzoyl hexyl benzoate.

A preferred solid organic UV(B) filter is a UV(B) filter which is selected from the group consisting of ethylhexyl triazone (=Uvinul® T150), diethylhexyl butamido triazone (=Uvasorb® HEB), and 4-methylbenzylidene camphor (=PARSOL® 5000).

The total amount of organic UV filter(s) depends strongly on the targeted UV protection.

It is preferred that the amount of a solid organic UV filter, particular of solid organic UV(A) filter, is selected in the range of 0.1 to about 6 wt.-%, preferable in the range of 0.5 to 5 wt.-%, most preferably in the range of 1 to 4 wt.-%.

It is further preferred that amount of a solid organic UV filter, particular of solid organic UV(B) filter, is selected in the range of 0.1 to about 6 wt.-%, preferable in the range of 0.5 to 5 wt.-%, most preferably in the range of 1 to 4 wt.-%.

It is even further preferred that amount of a liquid organic UV filter, particular of liquid organic UV(B) filter, is selected in the range of 0.1 to about 10 wt.-%, preferable in the range of 0.5 to 12 wt.-%, most preferably in the range of 1 to 10 wt.-%.

The cosmetic composition further preferably comprises at least one emulsifier, preferably an anionic emulsifier. Preferably the anionic emulsifier is an anionic emulsifier selected from the group consisting of potassium cetyl phosphate, disodium cetearyl sulfosuccinate, sodium stearoyl glutamate, sodium stearoyl lactylate, glyceryl stearate citrate and sodium cocoyl isethionate.

In one advantageous embodiment, the compositions in addition contain a phosphate ester emulsifier. Among the preferred phosphate ester emulsifier are C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-15 Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular preferred phosphate ester emulsifier is potassium cetyl phosphate e.g. commercially available as Amphisol® K at DSM Nutritional Products Ltd Kaiseraugst.

The cosmetic composition can also comprise nonionic emulsifiers.

Examples of nonionic emulsifier include condensation products of aliphatic (C8-C18) primary or secondary linear or branched chain alcohols with alkylene oxides, usually ethylene oxide and generally having from 6 to 30 ethylene oxide groups. Other representative nonionic emulsifiers include mono- or di-alkyl alkanolam ides such as e.g. coco mono- or di-ethanolamide and coco mono-isopropanolamide. Further nonionic emulsifiers which can be included are the alkyl polyglycosides (APGs). Typically, the APG is one which comprises an alkyl group connected (optionally via a bridging group) to a block of one or more glycosyl groups such as e.g. Oramix™ NS 10 ex Seppic; PLANTACARE® 818UP, PLANTACARE® 1200 and PLANTACARE® 2000 ex BASF.

If the cosmetic composition is an 0/W emulsion, then it preferably contains at least one 01W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten.

Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Another class of particularly suitable emulsifiers are polyglycerol esters or diesters of fatty acids also called polyglyceryl ester/ diester (i.e. a polymer in which fatty acid(s) is/ are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Also suitable are polyalkylenglycolether such as Brij 72 (Polyoxyethylen-(2)stearylether) or Brij 721 (Polyoxyethylene (21) Stearyl Ether e.g. available at Croda.

The at least one O/W respectively Si/W emulsifier is preferably used in an amount of 0.5 to 10 wt. % such as in particular in the range of 0.5 to 5 wt.-% such as most in particular in the range of 0.5 to 4 wt.-% based on the total weight of the composition.

Suitable W/O- or W/Si-emulsifiers are polyglyceryl-2-dipolyhydroxystearat, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, polygylceryl-4 oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weight of the composition.

The cosmetic compositions furthermore advantageously contain at least one co-surfactant such as e.g. selected from the group of mono- and diglycerides and/ or fatty alcohols. The co-surfactant is generally used in an amount selected in the range of 0.1 to 10 wt.-%, such as in particular in the range of 0.5 to 6 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The composition is preferably sulfate-free.

Hence, the cosmetic composition is preferably particularly free of sulfates of the group consisting of alkyl sulfates, alkyl ether sulfates, alkyl amido ether sulfates, alkylaryl polyether sulfates and monoglycerides sulfate as well as mixtures thereof.

The term “free” as used in the present document, for example in “sulfate-free”, is used to mean that the respective substance is only present at amounts of less than 0.5% by weight, particularly less than 0.1% by weight, more particularly below 0.05% by weight, relative to the weight of the composition. Preferably, “free” means that the respective substance is completely absent in the composition.

The term “sulfate-free” is used in the present document to mean that the composition is free of any anionic tenside having a terminal anionic group of the formula

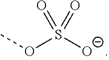

The cosmetic composition is preferably free of cationic emulsifiers. Typical example for such cationic emulsifiers are isostearamidopropyl dimethylamine, stearalkonium chloride, stearamidoethyl diethylamine, behentrimonium methosulfate, behenoyl PG-trimonium chloride, cetrimonium bromide, behenamidopropyl dimethylamine behenate, brassicamidopropyl dimethylamine, stearamidopropyl dimethylamine stearate, cocamidopropyl PG-dimonium chloride, distearoylethyl hydroxyethylmonium methosulfate, dicocoylethyl hydroxyethylmonium methosulfate, distearoylethyl dimonium chloride, shea butteramidopropyltrimonium chloride, behenamidopropyl dimethylamine, brassicyl isoleucinate esylate, acrylamidopropyltrimonium chloride/acrylates copolymer, linoleamidopropyl ethyldimonium ethosulfate, dimethyl lauramine isostearate, isostearamidopropyl laurylacetodimonium chloride, particularly behentrimonium chloride, distearyldimonium chloride, cetrimonium chloride, steartrimonium chloride, and palm itamidopropyltrimonium chloride.

The cosmetic composition further may comprise cosmetic carriers, excipients and diluents as well as additives and active ingredients commonly used in the skin care industry which are suitable for use in the cosmetic compositions are for example described in the International Cosmetic Ingredient Dictionary & Handbook by Personal Care Product Council

(http://www.personalcarecouncil.org/), accessible by the online INFO BASE
(http://online.personalcarecouncil.org/jsp/Home.jsp), without being limited thereto.

Such possible ingredients of the cosmetic composition are particularly enhance the performance and/or consumer acceptability such as preservatives, antioxidants, fatty substances/oils, thickeners, softeners, light-screening agents, moisturizers, fragrances, co-surfactants, fillers, sequestering agents, cationic-, nonionic- or amphoteric polymers or mixtures thereof, acidifying or basifying agents, viscosity modifiers, and natural hair nutrients such as botanicals, fruit extracts, sugar derivatives and/or amino acids or any other ingredients usually formulated into cosmetic compositions. The necessary amounts of the adjuvants and additives can, based on the desired product, easily be chosen by a person skilled in the art in this field and will be illustrated in the examples, without being limited hereto.

Particularly suitable thickeners in all embodiments are xanthan gum, gellan gum and/or carboxymethylcellulose. Most preferably in all embodiments the thickener is xanthan gum or gellan gum.

Such thickener(s) are preferably used in an amount (total) selected in the range from 0.1 to 1 wt.-%, more preferably in an amount of 0.1 to 0.5 wt.-%, based on the total weight of the cosmetic composition.

The cosmetic compositions have preferably a pH in the range from 3 to preferably a pH in the range from 4 to 8 and most preferably a pH in the range from 4 to 7.5. The pH can easily be adjusted as desired with suitable acids such as e.g. citric acid or bases such as NaOH according to standard methods in the art.

The cosmetic composition is preferably sulfate-free and/or free of parabens, and/or silicone oils and/or silicone surfactants.

The cosmetic composition is preferably a topical composition.

The term “topical” as used herein is understood here to mean external application to keratinous substances, which are in particular the skin, scalp, eyelashes, eyebrows, nails, mucous membranes and hair, preferably the skin.

As the topical compositions are intended for topical application, it is well understood that they comprise a physiologically acceptable medium, i.e. a medium compatible with keratinous substances, such as the skin, mucous membranes, and keratinous fibers. In particular, the physiologically acceptable medium is a cosmetically acceptable carrier.

The term “cosmetically acceptable carrier” refers to all carriers and/or excipients and/ or diluents conventionally used in cosmetic compositions such as in particular in sun care products.

Preferably the cosmetic composition is a skin care preparation, decorative preparation, or a functional preparation.

Examples of skin care preparations are, in particular, light protective preparations, anti-ageing preparations, preparations for the treatment of photoageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of functional preparations are cosmetic or pharmaceutical compositions containing active ingredients such as hormone preparations, vitamin preparations, vegetable extract preparations and/or anti-ageing preparations without being limited thereto.

The cosmetic composition is preferably a skin care composition.

In a most preferred embodiment, the cosmetic composition is a sun care composition. Sun care compositions are light-protective preparations (sun care products), such as sun protection milks, sun protection lotions, sun protection creams, sun protection oils, sun blocks or day care creams with a SPF (sun protection factor). Of particular interest are sun protection creams, sun protection lotions, sun protection milks and sun protection preparations.

The cosmetic compositions have improved sensory properties, particular improved afterfeel.

The cosmetic composition shows a significant lower rancidity, i.e. significantly longer induction time, compared to the respective composition without said ester of a fatty acid and dextrin and/or said mixture of branched and linear saturated C15-C19 alkanes.

The rancidity as used in the present document is quantified by the induction time measured by a Rancimat test as described in Laubli et al., JAOCS, 1986, 63(6), 792-795, using a 743 Rancimat apparatus (Methrom, Herisau, Switzerland).

It has been shown that by adding said mixture of branched and linear saturated C15-C19 alkanes as mentioned above to a vegetable oil, the rancidity was significantly reduced, i.e. the induction time was significantly expanded.

It has been further shown that by adding said ester of a fatty acid and dextrin as mentioned above to a vegetable oil the rancidity was significantly reduced, i.e. the induction time was significantly expanded. It has been observed that when adding said ester of a fatty acid and dextrin to the vegetable oil a higher reduction of rancidity, i.e. a longer induction time, has been observed as compared to adding the same amount of said mixture of branched and linear saturated C15-C19 alkanes to the vegetable oil.

Finally, it has been further shown that by adding said ester of a fatty acid and dextrin and said mixture of branched and linear saturated C15-C19 alkanes as mentioned above to a vegetable oil, the rancidity was significantly reduced, i.e. the induction time was significantly expanded. It has been observed that the rancidity was surprisingly most reduced, i.e. the induction time was most expanded, when both, said ester of a fatty acid and dextrin and said mixture of branched and linear saturated C15-C19 alkanes were added to the vegetable oil.

Hence, in a further aspect, the present invention relates to the use of adding an ester of a fatty acid and dextrin and/or a mixture of branched and linear saturated C15-C19 alkanes to reduce the rancidity of a vegetable oil.

Said ester and mixture of the of branched and linear saturated C15-C19 alkanes have been discussed already above in great detail.

This reduction of rancidity of said vegetable oils respectively of the cosmetic compositions comprising said vegetable oils by adding an ester of a fatty acid and dextrin and/or a mixture of branched and linear saturated C15-C19 alkanes is very surprising as neither said ester of a fatty acid and dextrin nor said mixture of branched and linear saturated C15-C19 alkanes are known to have any antioxidant effect.

The vegetable oil is particularly a vegetable oil which has an amount of unsaturated fatty acids of more than 35% by weight, particularly more than 40% by weight, in its triglycerides.

The vegetable oil is preferably selected from the group consisting of Macadamia integrifolia seed oil, Prunus amygdalus dulcis (sweet almond) oil, Argania spinosa kernel oil, Linum usitatissimum (linseed) oil, Perilla ocymoides seed oil, Butyrospermum parkii (shea) butter and Olea europaea (Olive) fruit oil.

It has been observed that the reduction of rancidity by said ester of a fatty acid and dextrin and/or said mixture of branched and linear saturated C15-C19 alkanes is most significantly, expressed as %-extension induction time, for Macadamia integrifolia seed oil, Butyrospermum parkii (shea) butter and Olea europaea (Olive) fruit oil.

The highest reduction of rancidity, expressed as %-extension induction time, has been observed for Macadamia integrifolia seed oil.

Due to the significant reduction of rancidity, a significantly extended shelf-life of the vegetable oil or the cosmetic composition, respectively, can be obtained by the present invention. This is of course very interesting for the end users as well as for the commercialisation of such a vegetable oil or cosmetic composition, respectively. It is further interesting to avoid adding any antioxidants to any cosmetic compositions comprising said vegetable oils.

Vegetable oils are healthy ingredients having powerful properties in cosmetics and are natural products. This is very important as the consumers and the market are more and more interested in sustainable ingredients and products for the fields of cosmetic and personal care. Therefore, it is important and interesting that vegetable oils can be used in cosmetics. However, as the odd smell of rancid oils can be detected already at very low concentrations, a reduction of the rancidity, particularly without adding any further antioxidants, as many of the antioxidants currently used are under discussion in view of undesired side effects. Therefore, the current invention is very advantageous on the one hand to extend the shelf-life of vegetable oils and their cosmetic products significantly and on the other hand also allowing the use of those vegetable oils which per se are very prone toward rancidity in cosmetic products.

### EXAMPLES

The present invention is further illustrated by the following experiments.

### Cosmetic Compositions and Rancidity

### Preparation of a Premix

The dextrin palmitate 1 and 2 have been premixed with a part of the C15-C19 alkane to form a pre-mix (Dextrin palm itate 25% by weight).

Said premix has then mixed with the respective vegetable oil and the rest of the C15-19 alkane by a magnetic stirrer at 80C, let cool down without any stirring to 25° C.

The rancidity of the compositions has been quantified by the induction time (Induction Time) measured at 110° C. by a Rancimat test as described in Laubli et al. Laubli et al., JAOCS, 1986, 63(6), 792-795, using a 743 Rancimat apparatus (Methrom, Herisau, Switzerland).

The measured induction time has been listed in tables 1 and 2.

Furthermore, the %-extension of induction time (Δ_{IT}) given for the compositions 1-11 are calculated as % change of the induction time of the respective composition in respect to the respective reference of pure vegetable oil used.

For example, the Δ_{IT} given in example 5 (+54.9%) in tables 1 and 2 are calculated from the induction times measured for example 5 and Ref.1 in the following way: Δ_{IT} =((7.56/4.88)-1)*100.

TABLE 1
Cosmetic compositions and rancidity
(all ingredients in % by weight).

| | Ref. 1 | 1 | 2 | 3 | 4 | 5 |
|---|---|---|---|---|---|---|
| Macadamia | 100 | 90 | 94 | 96 | 90 | 90 |
| integrifolia | | | | | | |
| seed oil [%] | | | | | | |
| C15-19 | | 10 | 6 | | | 6 |
| alkane [%]¹ | | | | | | |
| Dextrin | | | | 4 | 10 | |
| Palmitate | | | | | | |
| 1 [%]² | | | | | | |
| Dextrin | | | | | | 4 |
| Palmitate | | | | | | |
| 2 [%]³ | | | | | | |
| Induction | 4.88 | 5.44 | 5.67 | 5.91 | 6.27 | 7.56 |
| Time [h] | | | | | | |
| Δ_{IT} [%] | | +11.5% | +16.2% | +21.1% | +28.5% | +54.9% |

¹EMOGREEN ™ L19
²Rheopearl ® KL2, Chiba Flour Milling
³Dextrin Palmitate: Mₙ = 11′300-11′500 Da, determined by SEC/GPC

The examples of table 1 show that the mixture of branched and linear saturated C15-C19 alkanes (EMOGREEN™ L19) show a significant reduction of the rancidity of the vegetable oil (1 and 2 vs Ref. 1) as well as the ester of fatty acid and dextrin (dextrin palmitate) show a significant reduction of the rancidity of the vegetable oil (3 and 4 vs Ref.1). This reduction effect is even bigger for the C15-C19 alkanes than for the ester of fatty acid and dextrin (4 vs 1). Furthermore, the biggest reduction of rancidity is observed for a combination of mixture of branched and linear saturated C15-C19 alkanes (EMOGREEN™ L19) and the ester of fatty acid and dextrin (dextrin palmitate)(5 vs Ref.1).

The examples of table 2 give a comparison of the rancidity of different typical vegetable oils respectively their cosmetic compositions. The results of table 2 show that for all of these vegetable oils a significant reduction of rancidity (Δ_{IT}) could be observed. This reduction of rancidity is most pronounced for Macadamia integrifolia seed oil, followed by Butyrospermum parkii (shea) butter and followed by Olea europaea (Olive) fruit oil.

TABLE 2
Cosmetic compositions and rancidity (all ingredients in % by weight).

| | Ref. | | Ref. | | Ref. | | Ref. | | Ref. | | Ref. | | Ref. | |
|---|---|---|---|---|---|---|---|---|---|---|---|---|---|---|
| Vegetable oil | 1 | 5 | 2 | 6 | 3 | 7 | 4 | 8 | 5 | 9 | 6 | 10 | 7 | 11 |
| Macadamia | 100 | 90 | | | | | | | | | | | | |
| integrifolia | | | | | | | | | | | | | | |
| seed oil [%] | | | | | | | | | | | | | | |
| Butyrospermum | | | 100 | 90 | | | | | | | | | | |
| parkii | | | | | | | | | | | | | | |
| (shea) butter [%] | | | | | | | | | | | | | | |
| Europaea (Olive) | | | | | 100 | 90 | | | | | | | | |
| fruit oil [%] | | | | | | | | | | | | | | |
| Perilla | | | | | | | 100 | 90 | | | | | | |
| Ocymoides seed | | | | | | | | | | | | | | |
| oil [%] | | | | | | | | | | | | | | |
| Linum | | | | | | | | | 100 | 90 | | | | |
| usitatissimum | | | | | | | | | | | | | | |
| (linseed) oil [%] | | | | | | | | | | | | | | |
| Prunus amygdalus | | | | | | | | | | | 100 | 90 | | |
| dulcis(sweet | | | | | | | | | | | | | | |
| almond) oil [%] | | | | | | | | | | | | | | |
| Argania spinosa | | | | | | | | | | | | | 100 | 90 |
| kernel oil [%] | | | | | | | | | | | | | | |
| C15-19 alkane | | 6 | | 6 | | 6 | | 6 | | 6 | | 6 | | 6 |
| [%]¹ | | | | | | | | | | | | | | |
| Dextrin Palmitate | | 4 | | 4 | | 4 | | 4 | | 4 | | 4 | | 4 |
| 2 [%]² | | | | | | | | | | | | | | |
| Induction | 4.88 | 7.56 | 15.88 | 22.69 | 7.04 | 9.49 | 0.5 | 0.61 | 0.88 | 1.07 | 0.87 | 1.01 | 7.3 | 7.86 |
| Time [h] | | | | | | | | | | | | | | |
| Δ_{IT} [%] | | +54.9% | | +42.9% | | +34.8% | | +22.0% | | +21.6% | | +16.1% | | +7.7% |

¹EMOGREEN ™ L19
²Dextrin Palmitate: Mₙ = 11′300-11′500 Da, determined by SEC/GPC

1. A cosmetic composition comprising
an ester of a fatty acid and dextrin
a mixture of branched and linear saturated C15-C19 alkanes
a vegetable oil having an amount of unsaturated fatty acids of more than 35% by weight, particularly more than 40% by weight, in its triglycerides;
wherein the amount of branched saturated C15-C19 alkane in said mixture of branched and linear saturated C15-C19 alkanes is more than 80% by weight, preferably more that 90% by weight, most preferred more than 92% by weight.

2. The composition according to claim 1, wherein the amount of linear saturated C15-C19 alkanes in said mixture of branched and linear saturated C15-C19 alkanes is less than 10% by weight, preferably less than 8% by weight, most preferred more than 5% by weight.

3. The composition according to claim 1, wherein amount of branched saturated C18 alkane is more than 50% by weight, preferably more than 60% by weight, even more preferably more than 70% by weight, relative to the weight of said mixture of branched and linear saturated C15-C19 alkanes .

4. The composition according to claim 1 wherein the fatty acid of said ester of a fatty acid and dextrin is a C14-C18 fatty acid, particularly a linear C14-C18 fatty acid, most preferably palmitic acid.

5. The composition according to claim 1 wherein the dextrin of said ester of a fatty acid and dextrin has an average degree of glycopolymerization of between 3 and 20, particularly between 8 and 16.

6. The composition according to claim 1 wherein said ester of a fatty acid and dextrin has an average number of esterified hydroxyl groups of more than 3, preferably between 3.05 and 3.5, more preferably between 3.1 and 3.4, most preferably between 3.1 and 3.2 per glucose unit.

7. The composition according to claim 1 wherein said ester of a fatty acid and dextrin has an molecular weight Mₙ of between 8′000 and 16′000 g/mole, preferably between 9′000 and 13′000 g/mole, more preferably between 10′000 and 11′500 g/mole.

8. The composition according to claim 1 wherein the ratio of the weight of said ester of a fatty acid and dextrin to the weight of said mixture of branched and linear saturated C15-C19 alkanes is less than 100% by weight, preferably in the range of 50-80% by weight, most preferred in the range of 60-70% by weight.

9. The composition according to claim 1 wherein the ratio of the weight of mono-unsaturated fatty acids to the weight of unsaturated fatty acids in its triglycerides is more than 80%, by weight, preferably more than 85% by weight, most preferably more than 90% by weight.

10. The composition according to claim 1 wherein the vegetable oil is selected from the group consisting of Macadamia integrifolia seed oil, Prunus amygdalus dulcis (sweet almond) oil, Argania spinosa kernel oil, Linum usitatissimum (linseed) oil, Perilla ocymoides seed oil, Butyrospermum parkii (shea) butter and Olea europaea (Olive) fruit oil.

11. The composition according to claim 1 wherein the vegetable oil is selected from the group consisting of Macadamia integrifolia seed oil, Butyrospermum parkii (shea) butter and Olea europaea (Olive) fruit oil.

12. Use of adding an ester of a fatty acid and dextrin and/or a mixture of branched and linear saturated C15-C19 alkanes to reduce the rancidity of a vegetable oil.

13. Use according to claim 12, wherein the vegetable oil is having an amount of unsaturated fatty acids of more than 35% by weight, particularly more than 40% by weight, in its triglycerides.

14. Use according to claim 12, wherein the vegetable oil is selected from the group consisting of Macadamia integrifolia seed oil, Prunus amygdalus dulcis (sweet almond) oil, Argania spinosa kernel oil, Linum usitatissimum (linseed) oil, Perilla ocymoides seed oil, Butyrospermum parkii (shea) butter and Olea europaea (Olive) fruit oil.
